(19) United States
(12) Patent Application Publication
(10) Pub. No.: US 2007/0128136 A1
(43) Pub. Date: Jun. 7, 2007

(54) Terrein compound having melanin biosynthesis inhibitors and its preparation

(76) Inventors: Ick-Dong Yoo, Taejeon-si (KR); Won-Gon Kim, Taejon-si (KR); In-Ja Ryoo, Taejeon-si (KR); Jong-Pyung Kim, Taejeon-si (KR); Sangku Lee, Taejeon-si (KR); Seo-Hyoung Park, Seoul (KR); Dong-Seok Kim, Seoul (KR); Kyoung-Chan Park, Seoul (KR)

Correspondence Address: JHK LAW, P.O. BOX 1078, LA CANADA, CA, 91012-1078, US

(21) Appl. No.: 10/596,211

(22) PCT Filed: Oct. 19, 2004
(86) PCT No.: PCT/KR04/02677

§ 371 Date: Jun. 2, 2006

(30) Foreign Application Priority Data
Dec. 12, 2003 (KR) 10-2003-0090611

(51) Int. Cl. A61K 8/35; A61K 31/12

(52) U.S. Cl. 424/62; 514/690

(57) ABSTRACT

The present invention relates to a melanin biosynthesis inhibitor containing terrein compound as an effective ingredient. The terrein compound can be easily separated from Penicillium sp KCTC 26245, a fungal strain inhabited in domestic soil. It does not directly inhibit tyrosinase but inhibits the expression of MITF (microphthalmia-associated transcription factor) by activating ERK (extracellular signal-regulated kinase) in melanin chromatocytes to give whitening effect. So, the melanin biosynthesis inhibiting effect of the compound is much greater than that of any other conventional inhibitors, and further the effect can be raised when the compound is used together with other inhibitors, owing to their different mechanisms. Thus, the compound of the present invention can be effectively used as a skin trouble treating agent, a skin whitening agent and a browning inhibitor.

### TECHNICAL FIELD

The present invention relates to a novel use of a compound having melanin biosynthesis inhibiting activity that is originated from fungi and a preparation method of the same.

### BACKGROUND ART

Melanin is a biopolymer of phenols, which is of wide distribution in nature. It is a complex of black pigment and protein and has activity of increasing viability against any circumstances and competitiveness. However, over-production of melanin generates discoloration and freckle, resulting in skin aging or even skin cancer. Over-biosynthesis of melanin, resulted from severe exposure to UV owing to the destruction of ozone layer, does harm human, in particular skin. Thus, many attempts have been made to develop novel substances having a strong melanin biosynthesis inhibiting activity.

In regard to skin cancer, melanin biosynthesis has been a major target of study, from which various melanin biosynthesis inhibitors have been developed and applied to the fields of medicine, cosmetics and food industry, for example, to make a skin trouble treating agent, a skin whitening agent for the prevention and the treatment of discoloration and freckle, and a browning inhibitor, etc. And there is a great demand for the inhibitors in relation with environmental problems.

Factors involved in melanin biosynthesis are divided into two categories; one of them composed of factors directly inhibiting tyrosinase, a melanin biosynthesizing speed-regulating enzyme.

Tyrosinase is an enzyme that is combined with copper and is widely found in animals, plants, microorganisms and human. It accelerates aerobic oxidation in phenol compounds such as monohydroxy- or dihydorxy-phenylalanine (DOPA), etc, and induces skin damage and aging by depositing melanin pigment on skin being exposed on UV. In addition, polyphenoloxidase like tyrosinase causes browning reaction in food, in particular in vegetables or fruits.

Studies on melanin biosynthesis inhibitors have been focused on the development of a tyrosinase inhibitor, and representative tyrosinase inhibitors developed so far are substances forming chelate acting toward copper ion of tyrosinase active site, reducing agents like ascorbic acid converting quinones into phenols, and non-sulfite agents denaturating tyrosinase itself.

Tyrosinase inhibitors have been developed in various forms of whitening agent, which have been in used but have problems. For example, 4-hydroxyanisole and hydroquinine, which have been used locally for the treatment of hypermelanosis such as discoloration, freckle, spot, hyperpigmentation during gestation period, etc, have very strong melanin biosynthesis inhibiting activity but at the same time induce degeneration or even death of chromatocytes and damage original function of a cell. In particular, hydroquinone compounds, which have been developed as a whitening cream having melanin biosynthesis inhibiting activity, allegedly induces skin troubles or diseases resulted from their cytotoxicity, so that their use is permitted in only some countries.

The other factors involved in melanin biosynthesis do not directly inhibit tyrosinase but regulate a transcription factor involved in the expression of tyrosinase in melanin chromatocytes.

According to recent reports on ERK inhibition test, melanin biosynthesis in a melanin cell can be inhibited by ERK activation (Englaro W, et al. Inhibition of the mitogen-activated protein kinase pathway triggers B16 melanoma cell differentiation. J Biol Chem, 1998, 273:9966-9970) and MITF decomposition resulted from ERK activation (Wu M, et al. c-Kit triggers dual phosphorylations, which couple activation and degradation of the essential melanocyte factor Mi. Genes Dev 2000, 14:301-312).

Melanin biosynthesis inhibitors using such factors do not cause side effects accompanied with direct inhibition of tyrosinase, and further, when they are used together with conventional melanin biosynthesis inhibitors, they might rise the effect owing to their different mechanisms.

### DISCLOSURE

It is an object of the present invention to provide a melanin biosynthesis inhibitor that does not directly inhibit tyrosinase for whitening but inhibits the expression of MITF (microphthalmia-associated transcription factor) by accelerating ERK (extracellular signal-regulated kinase) activation and a preparation method of the same.

In order to achieve the above object, the present invention provides a melanin biosynthesis inhibitor containing a terrein compound as an effective ingredient.

In order to achieve the above object, the present invention also provides a method for separation of a terrein compound characteristically inhibiting melanin synthesis from Penicillium sp.

Although terrein compound of the present invention does not directly inhibit tyrosinase, it shows whitening effect by inhibiting the expression of MITF by accelerating ERK activation in melanin chromatocytes. In addition, the compound has little cytotoxicity, so that it does not cause significant side effects, comparing to conventional melanin biosynthesis inhibitors working toward tyrosinase directly, indicating that it is a promising candidate for a skin trouble treating agent, a skin whitening agent or a browning inhibitor.

### DESCRIPTION OF DRAWINGS

FIG. 1 is a schematic diagram showing the separation process of terrein compound from metabolite of Penicillium sp KCTC 26245,

FIG. 2 is a graph showing 1H-NMR (600 MHz) spectrum of the terrein compound of the present invention,

FIG. 3 is a graph showing HMBC (600 MHz) spectrum of the terrein compound of the present invention,

FIG. 4 is a graph showing the result of cytotoxicity test of the terrein compound of the present invention,

FIG. 5 is a graph showing the melanin biosynthesis inhibiting activity of the terrein compound of the present invention,

FIG. 6 is a graph showing the melanin biosynthesis inhibiting activity of kojic acid, a standard material,

FIG. 7 is a set of photographs showing the melanin biosynthesis inhibiting activity of the terrein compound of the present invention,

FIG. 8 is an electrophoresis photograph showing the effect of the terrein compound of the present invention on the expression of MITF and ERK signal transduction pathway.

### BEST MODEL

Hereinafter, the present invention is described in detail.

The present invention provides a melanin biosynthesis inhibitor containing a terrein compound represented by the following formula 1 as an effective ingredient.

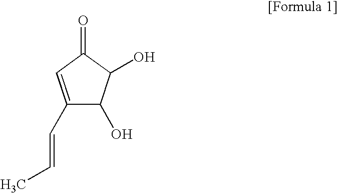

Terrein compound represented by formula 1 has a melanin biosynthesis inhibiting effect. As shown in FIG. 5-FIG. 7, like a conventional melanin biosynthesis inhibitor kojic acid, terrein compound of the present invention inhibits melanin biosynthesis dose-dependently, but the inhibiting effect of the compound of the invention is 10 times as strong as that of kojic acid.

Although terrein compound of the present invention does not inhibit tyrosinase directly, it characteristically inhibits the expression of MITF (microphthalmia-associated transcription factor) for whitening by accelerating ERK (extracellular signal-regulated kinase) activation in melanin chromatocytes. It has less side effects owing to low cytotoxicity than conventional melanin biosynthesis inhibitors that directly inhibit tyrosinase. Further, the whitening effect rises when it is used together with a conventional inhibitor owing to their different mechanisms. Thus, the compound of the present invention can be effectively used as a skin trouble treating agent, a skin whitening agent or a browning inhibitor.

The present invention also provides a method for the separation of terrein compound from Penicillium sp.

Precisely, the present invention provides a preparation method for terrein compound comprising the following steps;

Culturing Penicillium sp strain (step 1);

Obtaining the strain or its culture fluid from the above step 1 (step 2);

Obtaining ethyl acetate extract from the above strain or the culture fluid (step 3); and

Obtaining terrein compound of formula 1 by column chromatography with the ethyl acetate extract (step 4).

In step 1, Penicillium sp strain was cultured.

The Penicillium sp strain can be separated from soil. And Penicillium sp KCTC 26245 was preferably used herein. In step 1, Penicillium sp KCTC 26245 was cultured in yeast-malt extract medium (YM medium) at 28° C. with 140 rpm for 8 days.

After separating a novel strain KCTC 26245 from soil, melanin biosynthesis inhibiting effect of the strain was investigated to select the primary active strain. Among selected active strains that had highly activated active factors that could be extracted by ethyl acetate, a strain having the highest activity was selected. The selected strain was named as ‘Penicillium sp F020135’ and deposited at Korean Collection for Type Culture (KCTC) of Korea Research Institute of Bioscience and Biotechnology (KRIBB) on Aug. 24, 2003 (Accession No: KCTC 26245).

In step 2, a strain or culture fluid thereof was obtained. Particularly, a strain or culture fluid thereof containing metabolite of the strain was obtained from acetone extract of the cultured Penicillium sp KCTC 26245.

In step 3, ethyl acetate extract was obtained from the above strain or the culture fluid of the same. That is, aliquots were extracted by using ethyl acetate with the acetone extract obtained from the above strain or the culture fluid of the strain, which were then concentrated under decompression.

In step 4, column chromatography was performed to obtain terrein compound from the ethyl acetate extract obtained above.

Particularly, step 4 comprises the following two sub-steps;

Obtaining fractions by separating ethyl acetate extract prepared in step 3 through silica gel column chromatography using a mixed solvent of CHCl₃ and methanol as a moving phase (step 4-1); and

Obtaining terrein compound of the present invention by separating the fractions through sephadex-LH20 column chromatography using methanol as a moving phase (step 4-2).

In step (4-1), a mixed solvent of CHCl₃ and methanol was used as a moving phase, for which the mixing ratio of CHCl₃ to methanol was preferably 20:1-1:1.

In step (4-2), methanol was used as a moving phase. At that time, 100% methanol was preferably used for the first column chromatography, and then 70% methanol was preferably used for the second column chromatography.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of Terrein Compound of the Present Invention

A melanin biosynthesis inhibitor of the present invention was separated from the culture broth of Penicillium sp strain isolated from soil.

Particularly, Penicillium sp F020135 (Accession No: KCTC 26245), a fungal strain isolated from soil, was cultured on 100 ml of yeast-malt extract (YM) medium in 500 ml Erlenmeyer flask at 28° C. with 140 rpm for 8 days. Cells and culture solution were separated from each other by using a filter paper, and the cells were put in the same amount of acetone, which were left for overnight. The cells were discarded by filtering with a filter paper and acetone extract was obtained by concentration under decompression. The acetone extract and culture solution were put in the same amount of ethyl acetate for further extraction. The mixture was concentrated under decompression, resulting in ethyl acetate extract.

Fractions were obtained from the prepared ethyl acetate extract by silica gel column chromatography. At first, a mixed solvent of CHCl₃ and methanol was prepared at the ratio of 20:1 for elution. And the content of methanol in the mixed solvent was gradually increased to raise polarity until the ratio of 1:1. Among obtained fractions, those showing melanin biosynthesis inhibiting activity were selected.

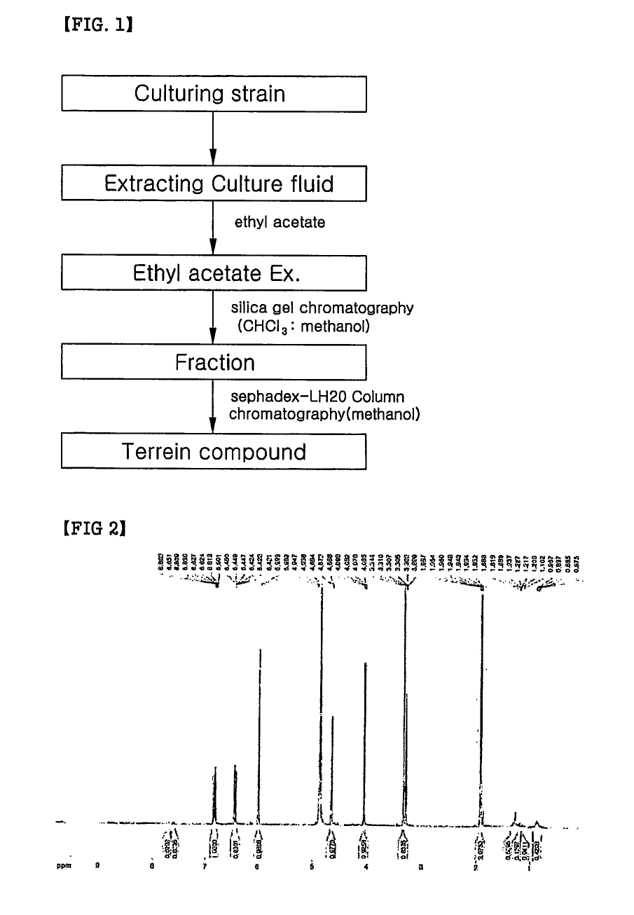

The obtained fractions were purified by sephadex LH-20 column chromatography that was saturated with 100% methanol solvent. At that time, methanol was used as an eluting solvent, and only active fractions were collected to obtain terrein compound of formula 1 (FIG. 1).

Physico-chemical property of the melanin biosynthesis inhibitor of the present invention.

Physico-chemical property of the compound of present invention, a melanin biosynthesis inhibitor, was investigated and as a result, it was confirmed to be white powder.

Mass analysis of the compound was also performed, and as a result, the molecular weight of the compound was confirmed to be 154. Based on the above results along with the result of 1H, 13C NMR spectrum data analysis, the molecular formula of the compound of the present invention was determined as C₈H₁₀O₃.

Chemical structure of the compound of the present invention.

In order to identify the chemical structure of the compound, one-dimensional NMR including 1H NMR spectrum and 13C NMR spectrum, and two-dimensional NMR, for example HMBC spectrum, were performed.

The compound of the present invention was dissolved in deuterium methanol (CD3OD) to investigate 1H NMR spectrum and 13C NMR spectrum. As a result, it was confirmed to be a terrein compound having chemical structure of 4,5-dihydroxy-3-propenyl-2-cyclopenten-1-one represented by formula 1.

After NMR data analysis and 13C NMR spectrum investigation, 8 carbon peaks were observed at 19.2 (CH3), 78.0 (CH), 82.5 (CH), 125.0 (CH), 126.1 (CH), 145.0 (CH), 170, and at 205.5 ppm. From 1H-NMR spectrum investigation, other peaks were also observed at 1.94 ppm (3H, dd, J=6.9, 1.5 Hz), 4.07 ppm (1H, d, J=2.7 Hz), 4.67 ppm (1H, d, J=2.7 Hz), 6.0 ppm (1H, s), 6.44 ppm (1H, dd, J=15.6, 1.5), and at 6.81 ppm (1H, aq, J=6.9, 15.6). The structure of the compound was finally determined as that of terrein of formula 1, based on the investigation of molecular weight and the result of NMR data analysis.

### MANUFACTURING EXAMPLE 1

### Preparation of Cream Containing Terrein Compound of the Present Invention

Stearic acid, cetostearyl alcohol, carlyric/capric triglyceride, mineral oil and butylene glycol were put together in a beaker in a water bath, which was heated to 75° C. to make oil phase. Terrein compound prepared in the above example 1, water, glycerin, tween 60, tween 80 and potassium hydroxide were mixed, resulting in aqueous phase, into which the above oil phase was added. The reaction solution was stirred with 1200 -1500 rpm for 10-20 minutes, and then cooled down. The solution was left at room temperature for 1-2 days. Contents of the included materials in the cream were presented in the below table 1 (total weight was 100 g).

TABLE 1

| Material | Amount (g) |
|---|---|
| Terrain compound | 0.1 |
| Stearic acid | 3.0 |
| Cetostearyl alcohol | 2.0 |
| Butylene glycol | 3.0 |
| Mineral oil | 8.0 |
| Carlyric/capric triglyceride | 3.0 |
| Glycerin | 6.0 |
| Tween 60 | 2.5 |
| Tween 80 | 1.0 |
| Potassium hydroxide | 0.5 |
| Distilled water | Proper amount |

### EXPERIMENTAL EXAMPLE 1

### Cytotoxicity Test of the Compound of the Present Invention

In order to investigate cytotoxicity of terrein compound of the present invention, Mel-Ab cells were treated with the terrein compound.

Particularly, Mel-Ab melanin cells were cultured in DMEM medium supplemented with 10% fetal bovine serum (FBS), 100 nM phobol 12-myristate 13-acetate, 1 nM cholera toxin, 50 ug/ml of streptomycin and 50 U/ml of penicillin under the condition of 5% CO₂, 37° C.

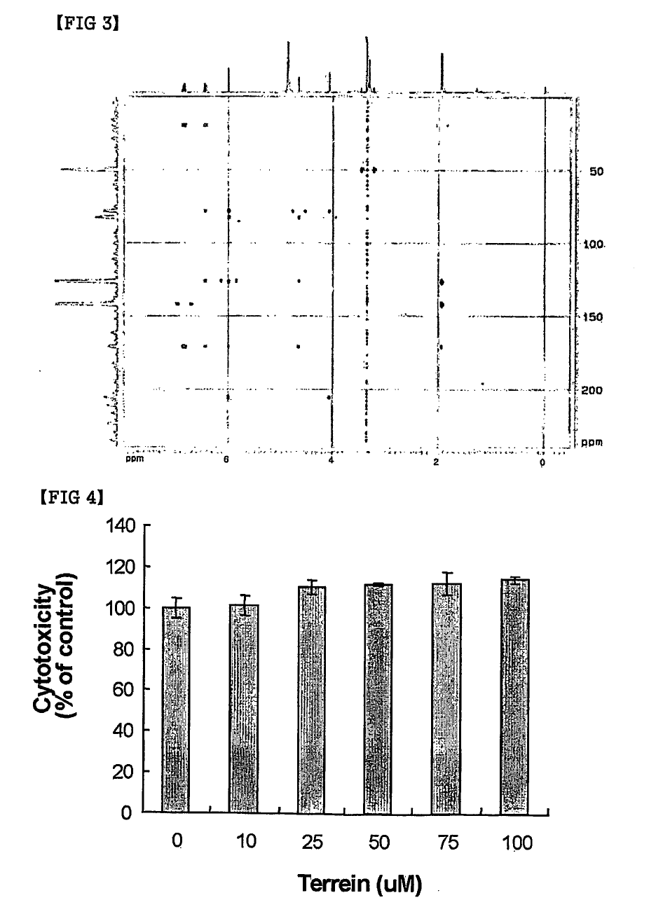

Mel-Ab melanin cells cultured above were treated with terrein compound at different concentrations (0, 10, 25, 50, 75, 100 uM) for 24 hours, followed by further culture. Then, medium was removed. Cells were stained with 0.5 ml of 0.1% crystal violet and Mel-Ab melanin cells were checked. Crystal violet was removed by washing several times. Crystal violet remaining in cells was extracted by using 1.0 ml of 95% ethanol. OD₅₉₀ of the extracted crystal violet was measured by ELISA reader in order to investigate vitality of cells. And the result was shown in FIG. 4.

As shown in FIG. 4, terrein compound of the present invention showed no cytotoxicity under the concentration of 100 uM, indicating that the compound can be safely administered to human.

### EXPERIMENTAL EXAMPLE 2

### Melanin Biosynthesis Inhibiting Effect of the Compound of the Present Invention

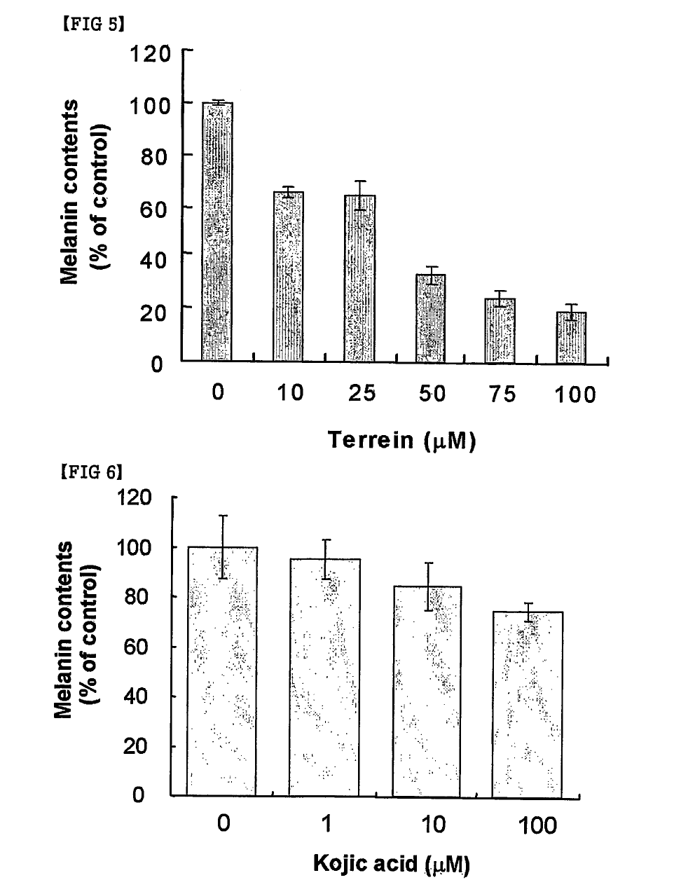

Mel-Ab melanin cells cultured in the above experimental example 1 were treated respectively with terrein compound and kojic acid, a conventional whitening agent, at different concentrations (0, 10, 25, 50, 75 and 100 uM of terrein compound and 0, 1, 10 and 100 uM of kojic acid). Then, melanin biosynthesis resulted from each treating was compared and the results were shown in FIG. 5 and FIG. 6.

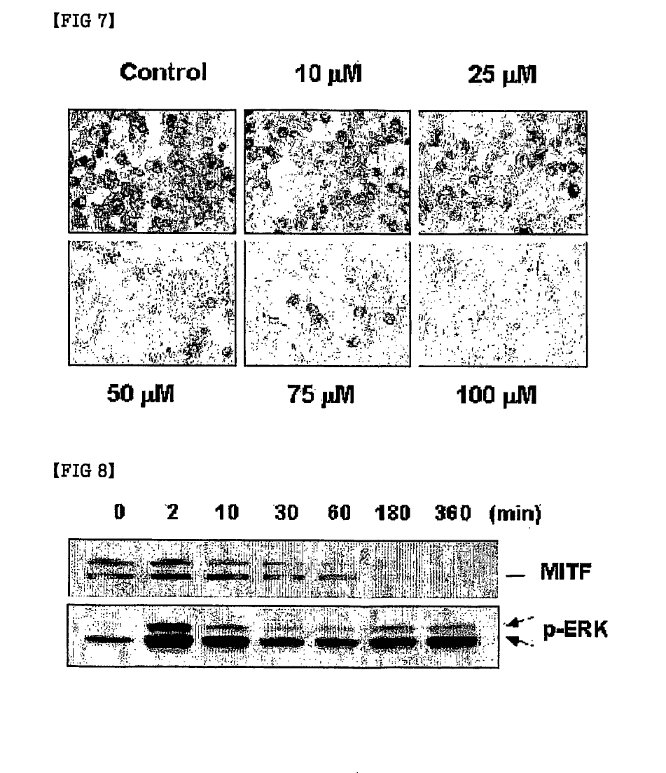

Both Mel-Ab melanin cells cultured under general culture conditions and Mel-Ab melanin cells treated with terrein compound of the present invention were observed under a microscope for comparison, and the result was shown in FIG. 7.

As shown in FIG. 5 and FIG. 6, terrein compound and kojic acid inhibited melanin biosynthesis dose-dependently. However, melanin biosynthesis inhibiting effect of terrein compound at the concentration of 10 uM was 10 times greater than that of kojic acid at the same concentration.

As shown in FIG. 7, dark melanin of Mel-Ab melanin cells cultured under normal conditions was clearly decreased by the treatment of terrein compound dose-dependently, indicating that terrein compound inhibits melanin biosynthesis in Mel-Ab melanin cells.

### EXPERIMENTAL EXAMPLE 3

### ERK Activation and MITF Decomposition in Mel-Ab Melanin Cells

In order to investigate melanin biosynthesis inhibiting mechanism of terrein compound of the present invention, Mel-Ab melanin cells cultured above were treated with 100 uM of terrein compound. And at each time point of 0, 2, 10, 30, 60, 180, and 360 minute, ERK (extracellular signal-regulated kinase) activation and MITF (microphthalmia-associated transcription factor) decomposition were investigated by Western blot. The results were shown in FIG. 8.

As shown in FIG. 8, after 2-10 minutes from the treatment of terrein compound to Mel-Ab melanin cells, ERK1 and ERK2 were activated fast and the activation was continued at least for 6 hours. But, the expression of MITF was decreased with ERK activation.

According to recent reports on ERK inhibition test, melanin biosynthesis in a melanin cell can be inhibited by ERK activation (Englaro W, et al. Inhibition of the mitogen-activated protein kinase pathway triggers B16 melanoma cell differentiation. J Biol Chem, 1998, 273:9966-9970) and MITF decomposition resulted from ERK activation (Wu M, et al. c-Kit triggers dual phosphorylations, which couple activation and degradation of the essential melanocyte factor Mi. Genes Dev 2000, 14:301-312).

In conclusion, as shown in FIG. 8, terrein compound included in a whitening composition of the present invention activates ERK, resulting in the regulation of the expression of MITF, which indicates that the compound has whitening effect by inhibiting melanin biosynthesis in melanin cells.

### INDUSTRIAL APPLICABILITY

As explained hereinbefore, the present invention relates to a melanin biosynthesis inhibitor containing terrein compound as an effective ingredient. The terrein compound of the present invention can be easily separated from Penicillium sp KCTC 26245, a fungal strain inhabited in domestic soil. It does not directly inhibit tyrosinase but inhibits the expression of MITF by activating ERK in melanin chromatocytes to give whitening effect. So, the melanin biosynthesis inhibiting effect of the compound is much greater than that of any other conventional inhibitors, and further the effect can be raised when the compound is used together with other conventional inhibitors, owing to their different mechanisms. Thus, the compound of the present invention can be effectively used for the production of a skin trouble treating agent, a skin whitening agent and a browning inhibitor.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A melanin biosynthesis inhibitor containing terrein compound represented by the following formula 1 as an effective ingredient.

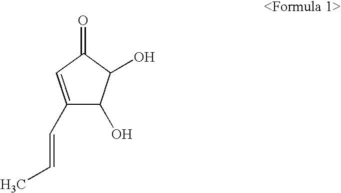

2. The melanin biosynthesis inhibitor as set forth in claim 1, wherein the melanin biosynthesis inhibitor is used as a skin trouble treating agent.

3. The melanin biosynthesis inhibitor as set forth in claim 1, wherein the melanin biosynthesis inhibitor is used as a skin whitening agent.

4. The melanin biosynthesis inhibitor as set forth in claim 1, wherein the melanin biosynthesis inhibitor is used as a browning inhibitor.

5. A preparation method for terrein compound comprising the following steps:
Culturing Penicillium sp strain KCTC 26245 (step 1);
Obtaining the strain or its culture fluid from the above step 1 (step 2);
Obtaining ethyl acetate extract from the above strain or the culture fluid (step 3); and
Obtaining terrein compound of claim 1 by column chromatography with the ethyl acetate extract (step 4).

6. The preparation method as set forth in claim 5, wherein the step 4 comprises the following two sub-steps:
Obtaining fractions by separating ethyl acetate extract prepared in step 3 through silica gel column chromatography using a mixed solvent of CHCl₃ and methanol as a moving phase (step 4-1); and
Obtaining terrein compound of the present invention by separating the fractions through sephadex-LH20 column chromatography using methanol as a moving phase (step 4-2).
